(19) United States
(12) Patent Application Publication
(10) Pub. No.: US 2024/0197290 A1
(43) Pub. Date: Jun. 20, 2024

(54) ULTRASONIC SURFACE OPTIMIZER

(71) Applicant: Enrique Rangel Gonzalez, Coyoacan (MX)

(72) Inventor: Enrique Rangel Gonzalez, Coyoacan (MX)

(21) Appl. No.: 18/219,900

(22) Filed: Jul. 10, 2023

(30) Foreign Application Priority Data
Dec. 15, 2022 (MX) MX/U/2022/000597

(51) Int. Cl. A61B 8/00

(52) U.S. Cl. CPC A61B 8/4455

(57) ABSTRACT

The present invention refers to an ultrasonic surface optimizer or ultrasonic probe to which a rough texture has been added to the entire tip of the sonotrode, which constitutes an improvement not only in its design but also in process efficiency, since it allows fragmenting or emulsify subcutaneous soft tissues optimally. The probe contains the following components: an ultrasonic vibrational energy source, a longitudinal handle with a proximal end and a distal end, each with a longitudinal stem axis. A connection at the proximal end of the longitudinal stem for connecting the longitudinal stem to the ultrasonic vibratory energy source; a point at the distal end of the longitudinal shank and one or more grooves near the tip, the grooves being generally transverse to the axis.

### TECHNICAL FIELD OF THE INVENTION

The present invention belongs to the field of invasive medical devices that are intended for short-term use, specifically for class II and class III classification, according to the level of risk associated with their use. From this classification, it is considered an active medical device whose operation depends on a source of electrical energy and acts as a conversion of said energy.

It also refers to surgically invasive medical devices such as an ultrasonic surface enhancer as it penetrates into the body through the body surface by means of a surgical procedure.

### BACKGROUND OF THE INVENTION

The present invention relates to the area of surgical instruments, particularly surgical-type medical devices that come into contact with soft tissue, such as an ultrasonic surface optimizer.

The surgical intervention known as liposuction or liposculpture is based on the extraction of excess adipose tissue, a procedure that requires a cannula that is introduced through incisions in the skin to tear or crush the adipose tissue, which is then removed thanks to a suction method.

The cannulas used for the adipose tissue detachment procedure are characterized by being small-diameter metal tubes, with closed and rounded tips. They are inserted into the patient through incisions in the skin and then the surgeon performs continuous movements of the cannula, which induces the removal of adipose tissue, as well as the disruption of other tissues; which causes irregularities, bruising, bleeding, swelling and numbness as side effects of said procedure.

Ultrasound-assisted liposuction (UAL) technique, for its acronym in English (Ultrasonic Assisted Liposuction) is used particularly in the liposuction procedure and works as follows:
a) High-frequency ultrasonic sound waves are used to obtain a homogenized mixture, which are transmitted through a solid cannula-type tube called a sonotrode and which allows the emulsification process of adipose tissue to be carried out.
b) The Sonotrode is introduced through small incisions made in a subdermal plane to break up the tissues and detach the fat cells.
c) On the other hand, the power source assists a transducer that converts the energy and transmits it to the sonotrode to have mechanical vibrations at the same frequency.

In the state of the art there are several patents for the invention of instruments intended to improve the liposuction or lipoplasty process, as it is also known, ultrasound-assisted (UAL) for the removal of adipose tissue, which we describe below:
I. US patent document U.S. Pat. No. 5,255,669A with the title: Ultrasonic Treatment Apparatus, with a publication date of Oct. 26, 1993, which refers to an apparatus consisting of a probe that has a system for generating ultrasonic vibrations and a sheath to cover at least one outer peripheral surface of the vibrational transmission member of the ultrasonic probe, plus a fluid supply unit adjacent to a coupled portion of the sheath for supplying a fluid within a defined passage between the vibrational transmission member and the sheath. Since it doesn't mention a sonotrode with a rough texture, it can be inferred that it is a different invention from the one proposed by the ultrasonic surface optimizer of the present invention.
II. Patent document US2018/0021599 A1, which refers to an apparatus for removing fat by separating lipocytes, which uses ultrasonic waves for this purpose. May include: a fat removal body part; a handpiece including an ultrasonic wave generating unit electrically connected to the fat removal body part; a tip portion provided on the handpiece: a temperature sensor unit for detecting the temperature of the tip, the temperature sensor unit being provided in a tip part; an interlocked pump part for supplying cooling water to the tip, the interlocked pump part being disposed in the grease removal body unit and being connected to the handpiece; and a controller for receiving a signal detected by the temperature sensor unit, and operating the locked pump part at a preset temperature. Although you may consider some other devices related to the fat removal apparatus, it does not mention a sonotrode with rings with a rough texture as in the present invention.
III. Spanish patent document number ES2209119, which discloses an ultrasonic surgical device (80, 180) that includes an adapter transducer assembly (82, 182) to vibrate at an ultrasonic frequency, in response to electrical energy. A transmission component (86, 186) adapted to receive ultrasonic vibration from the transducer array and transmit ultrasonic vibration from a first end to a second end. A braking device (130, 300) configured to apply a force to the outer surface of the drive component to damp ultrasonic vibration transmitted from the first end to the second end of the drive rod. Also provided is a method for inserting a trocar cannula into a patient's tissue. The procedure includes the steps of providing an ultrasonic trocar obturator (180), having an ultrasonic waveguide (186), including an effector (188) at the distal end thereof, positioning the trocar obturator within a cannula (159), placing the trocar plug in contact with a wall of said body cavity, and ultrasonic vibration of the end effector to create a penetration opening. The method further includes advancing the ultrasonic plug (180) into said penetration opening, sensing when penetration is complete, applying the braking process (300) to the plug to damp ultrasonic vibration of the end effector, and advancing the trocar cannula into the penetration opening, thus being different from the object of protection found in the present invention.
IV. U.S. Pat. No. 6,368,299 B1 to William W. Cimino published Apr. 9, 2000, which refers generally to an ultrasonic probe for enhanced fragmentation consisting of a longitudinal stem having a proximal tip, a distal tip and a shaft. The shaft joins the proximal end and the distal end. The proximal end of the longitudinal stem has a longitudinal stem connector attached to it by an ultrasonic vibratory energy source, (FIG. 1) The distal end of the longitudinal stem may have a rounded or pointed tip. The shaft has one or more grooves near the tip that circumscribe the axis of the longitudinal shank. The walls of one or more slots in the axis of the longitudinal stem provide a surface area at the distal end of the ultrasonic probe in planes generally perpendicular to the axis of the ultrasonic probe. (FIG. 2) The additional surface area increases the tissue fragmentation surface area of the distal end of the ultrasonic probe without increasing the diameter of the distal end of the ultrasonic probe. Thus, one is able to more quickly and completely fragment or emulsify tissues with a given diameter from the distal end of the ultrasonic probe. In addition, because surface area that gets in contact with the tissue along and around the distal end of the ultrasonic probe is reduced, the potential for tissue burns is also reduced. However, they have certain limitations, since the diameter of the cannula is increased, larger incisions must be made in the patient or a very pronounced low relief is made to create the rings, causing the structure of the cannula to destabilize with risk of being cut off.

Using the results obtained with the previous methods as a reference, an improvement was made to this medical device that seeks the emulsification of fat, reducing thermal shock, reducing the risks of burns and other complications in patients undergoing this procedure.

In the present invention, the issue of increasing surfaces is addressed as an aspect to be explored to improve the procedure and consequently its versatility in time and cost, minimizing the current problems of overheating.

Therefore, the present invention refers to an ultrasonic surface optimizer that is a medical device for surgical use that contains a generator with an elongated cannula-type sonotrode provided with micro surfaces located at the end of the cannula, which allows the most efficient application in the output of ultrasound (UAL), reducing the thermal impact towards the tissue of the human body, in addition to reducing the risks of burns and complications in patients undergoing this procedure, where this elongated cannula assists in the procedure to emulsify and homogenize fat by through an ultrasonic frequency (UAL), which is introduced into the human body through small incisions, where coordinated movements are made by the operator, to propagate the waves and break and detach the fat cells. GOALS

In first place, the present invention aims to provide a medical device for surgical use that contains a generator with an elongated cannula-type sonotrode, provided with micro surfaces located at the end of the cannula, which allows the efficient application of ultrasound output (UAL), reducing the thermal impact towards the tissue of the human body. In addition to reducing the risks of burns and complications in patients undergoing this procedure.

Likewise, another object of the present invention is to provide an ultrasound-assisted medical device, which optimizes the ultrasound output (UAL) by creating microsurfaces at one end of the cannula, by different elements at different scales.

Finally, it is sought that said sonotrode, elongated cannula type, assist in the procedure to emulsify and homogenize the fat by means of an ultrasonic frequency (UAL) through smaller incisions and where through the coordinated movements of the operator it is possible to break and break down the fat cells.

### DESCRIPTION OF THE INVENTION

### Brief Description of the Figures

FIG. 1 represents a side view and the effect of ultrasound is perceived at the end of the cannula, it is observed that the shading is the part designated for surface finish and enhancement for ultrasound-assisted liposuction (UAL).

FIG. 2 represents a frontal view, the emission of energy proportional to 360° is perceived.

FIG. 3 represents a detail view of ultrasound or ultrasound-assisted liposuction (UAL) output due to repetitive surface deviations in surface finish undulations.

FIG. 4 shows the detail of the microscopic irregularities of the real surface, the length scales are perceived in the undulations and in the roughness.

FIGS. 5a-5e represent the details of the orientation variations that a textured surface can have, which are categorized respectively as: 1) Perpendicular to the direction of the grooves, 2) According to the bisector of the angles formed by the directions of the grooves, 3) In any direction, 4) Radial and 5) Normal to a radius.

FIG. 6 shows a series of photographs comparing a horn without the optimization surface vs a horn with the optimization surface.

FIG. 7 shows a magnified image of a smooth sonotrode surface Vs a magnified image of a rough sonotrode surface.

FIG. 8 represents how by applying the roughness model at the end of the sonotrode, it is possible to make active areas the areas that are not commonly used, where a series of peaks and valleys can be observed that by their nature show angles along the length of the sonotrode wherever there is a rough surface, taking the entire face of any peak or valley that is transversal to the longitudinal axis of the sonotrode, as an active surface as a striking surface or ultrasound output.

FIG. 9 represents a sonotrode without a rough surface.

FIG. 10 represents a sonotrode with a rough surface.

FIGS. 11 and 12 represent the connection of the two equipment connected to the direct current plug located in the same room and the connection of the Heuss equipment where there is a cable attached to a handpiece or handle.

FIG. 13 represents how the twenty-seven centimeters long titanium cannula is attached, and then the before mentioned device was turned on by operating the power switch located on its rear part, when turning on the device, on a screen that is located on its front part. Various images appeared, including a counter or stopwatch at zero and a power indicator at 100 level.

FIG. 14 represents how the counter is activated by pressing a pedal or external trigger and immediately afterwards it began to repeatedly rub the titanium cannula with roughness on a previously moistened chamois cloth and placed spread out on the same table, it continued with this friction action until the counter marked one minute.

FIG. 15 represents a Fluke VT04 model thermal imaging camera, serial number 20100324.

FIG. 16 shows how they turned on the Fluke model VT04 thermographic camera, serial number 20100324, according to the inscriptions attached to its surface, which was placed at a distance of approximately thirty centimeters aligned on the central part of the cannula with a rough surface that had been rubbed and after a few seconds in the aforementioned camera appeared the image of a number indicating 43.3° C.

FIG. 17 represents how the device is turned on again and proceeds in the same way, for which the counter was activated by pressing a pedal or external trigger and immediately afterwards he began to repeatedly rub the titanium cannula without roughness on a damp chamois cloth, previously and placed extended on the same table, it continued with this friction action until one minute was reached.

FIGS. 18 and 19 show that the counter was activated by pressing a pedal or external trigger and immediately afterwards it began to repeatedly rub the titanium cannula without roughness on a previously moistened chamois cloth and placed flat on the same table, it continued with this action of friction until it marked one minute, at which point it is suspended and turned on the Fluke VT04 thermographic camera again, according to the inscriptions attached to its surface. Which was placed at a distance of approximately 30 centimeters, aligned over the central part of the cannula that had been rubbed, and after a few seconds in the mentioned camera the image of a figure indicating 45.5° C. appeared.

FIG. 20 represents the smooth sonotrode in operation with thinner, where it shows that the sonotrode in operation makes several dispersed bubbles, which does not generate a large active area that optimizes the ultrasound output.

FIG. 21 represents the rough sonotrode, where it shows its operation in the form of a bubble that arises at the tip and goes downwards, which generates a very large active area that optimizes the ultrasound output, which makes it have a low sound.

### DETAILED DESCRIPTION OF THE INVENTION

The present invention refers to an ultrasonic surface optimizer that is a medical device for surgical use that contains a generator with an elongated cannula-type sonotrode that assists in the procedure to emulsify and homogenize fat by means of ultrasonic frequency or ultrasound-assisted liposuction. (UAL), which is introduced into the human body through small incisions, where coordinated movements are made by the operator, to propagate the waves, break and gradually detach the fat cells.

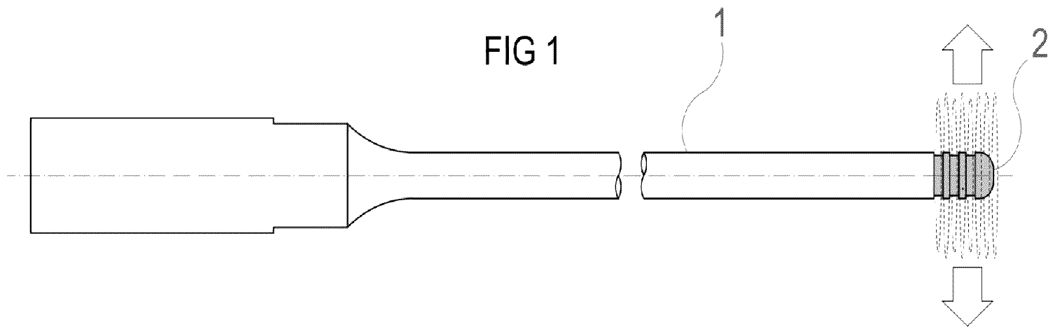

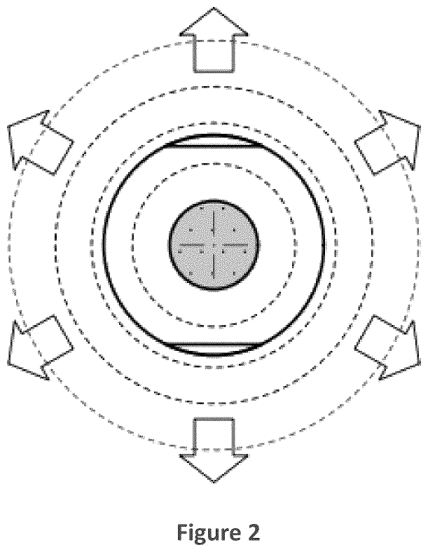

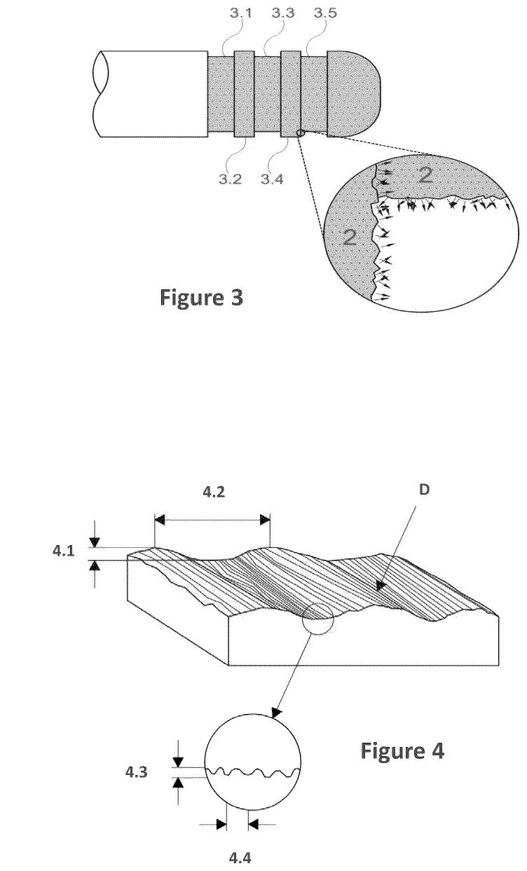

It is made up of an elongated cannula-type sonotrode, as shown in FIG. 1, at the end of the cannula there are a plurality of grooves (3.1, 3.3, 3.5), which create a series of surfaces of different levels such as rings (3.2, 3.4) as shown in FIG. 3, which direct energy proportional to 360°, as shown in FIG. 2.

More specifically, the present invention relates to an ultrasonic surface optimizer or an ultrasonic probe powered by an ultrasonic vibrational energy source and allowing fragmentation and emulsification of subcutaneous soft tissues.

The tip of the present invention has a special surface finish, designed in order to potentiate the output of ultrasound, doing the following process:

As shown in FIG. 1, which consists of repetitive deviations formed by undulations and which in turn are made up of peaks and valleys, as shown in FIG. 4, that is, it is a set of irregularities that are formed on a different scale, of length. On the first scale we have the undulation deviations and this in turn is made up of roughness deviations made up of peaks and valleys. This composition creates micro surfaces that generate an optimal space for the effect of mechanical hammering of ultrasound (UAL), that is to say that the density of the energy is optimized along the constructed surfaces thanks to the increase of the surface area in planes with different orientations and in different scales, such as a corrugation height (4.1), a corrugation spacing (4.2), both with a certain direction (D), and where the roughness presents a certain height (4.3) and a certain width (4.4).

In addition, as already mentioned above, U.S. Pat. No. 6,368,299 B1 refers to an ultrasonic probe to fragment or emulsify soft tissues, the ultrasonic probe powered by an ultrasonic vibrational energy source, the ultrasonic probe comprises the following elements:
Longitudinal handle with a proximal end and a distal end.
Axis of the longitudinal stem that joins the proximal end and the distal end.
Axis of the longitudinal stem aligned with the center of the longitudinal shank and passing through the proximal end and the distal end;
Connection at the proximal end of the longitudinal stem for connecting the longitudinal stem to the source of ultrasonic vibratory energy.
Tip at the distal end of the longitudinal shank, and one or more grooves near the tip.

Grooves generally transverse to the axis, in the axis of the longitudinal, and substantially circumscribing the axis of the longitudinal shaft, thereby reducing the tissue contact surface along and around the sides of the tip and providing a tissue comminution surface additional tip in planes generally perpendicular to axis; wherein the one or more slots each have a bottom, each bottom, in a cross section containing the axis of the longitudinal stem, which is substantially flat and substantially parallel to the longitudinal axis. Each bottom of one or more grooves is U-shaped in cross section containing the axis of the longitudinal stem. One or more slots do not completely circumscribe the shaft.

The longitudinal stem is solid and hollow and has a lumen open along its length, aligned with the axis of the longitudinal stem, and located generally in the center of the longitudinal stem. A conical section is formed in the longitudinal stem positioned along the axis of the longitudinal stem and between the axis of the longitudinal stem and its distal end, the tapered section being such that the diameter of the longitudinal stem and the distal end of the longitudinal stem decreases. The tip at the distal end of the longitudinal stem is blunt-shaped or bullet-shaped.

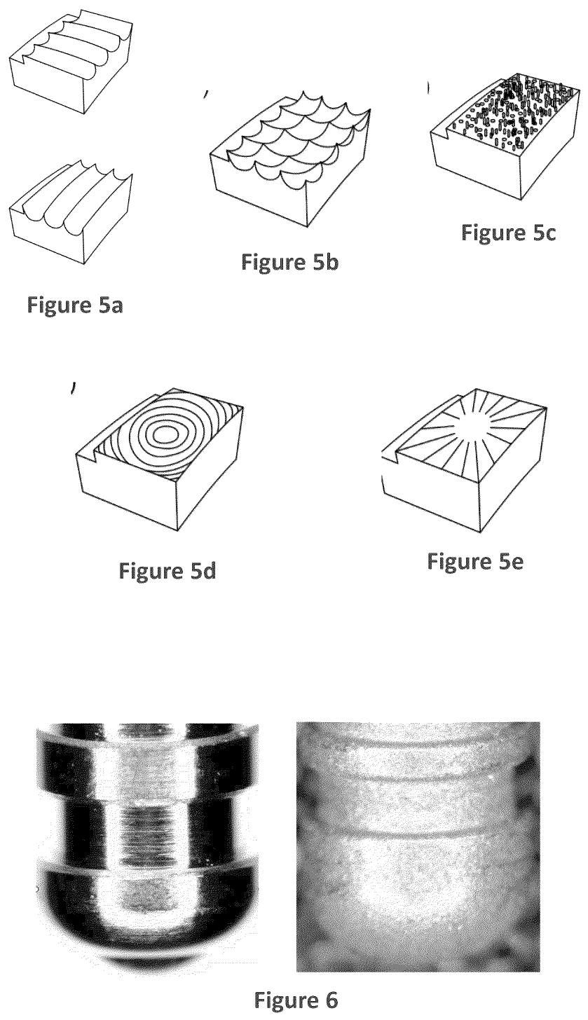

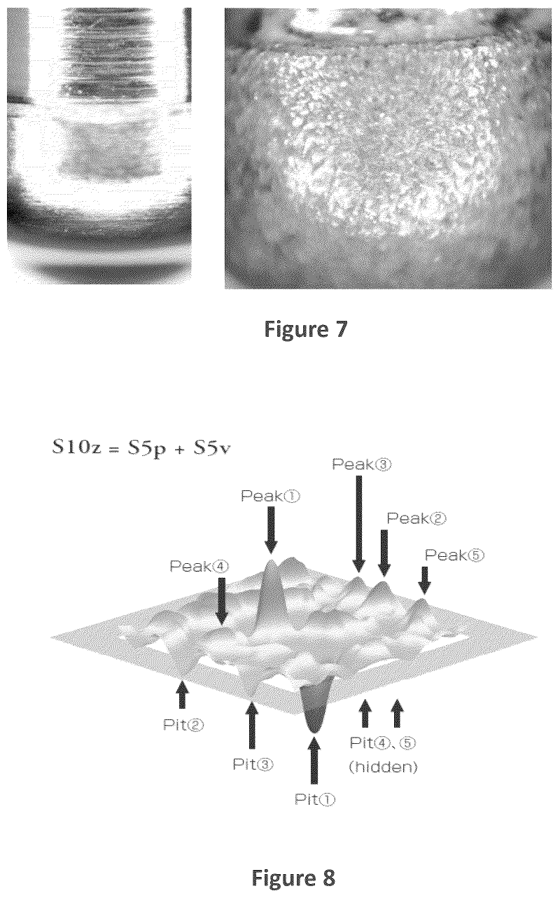

The present invention refers to the use of roughness and how this characteristic improves the action of the ultrasonic surface optimizer to fragment or emulsify the soft tissues of a patient, since the roughness makes it cover the different orientations that a texture can have, this direction or pattern, as shown in FIG. 5, is categorized into:
1) Perpendicular to the direction of the grooves as illustrated in FIG. 5a.
2) According to the bisector of the angles formed by the directions of the furrows as illustrated in FIG. 5b.
3) In any direction as illustrated in FIG. 5c.
4) Radial as illustrated in FIG. 5d.
5) Normal to a radius as illustrated in FIG. 5e.

The rough finish offers an advantage over other devices with a smooth or only grooved finish, since through the arrangement of this micro-texture formed by peaks and valleys, the longitudinal surfaces to the axis of the sonotrode are optimized, which are not used with the smooth or simply grooved model. In addition, any transversal face that is in contact with the peaks and valleys that function as an extender of the longitudinal and angular surface will receive the action of the ultrasonic wave.

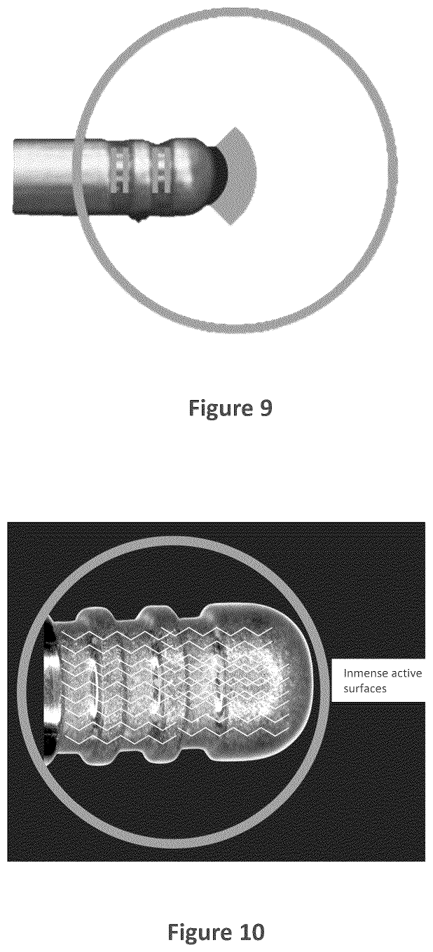

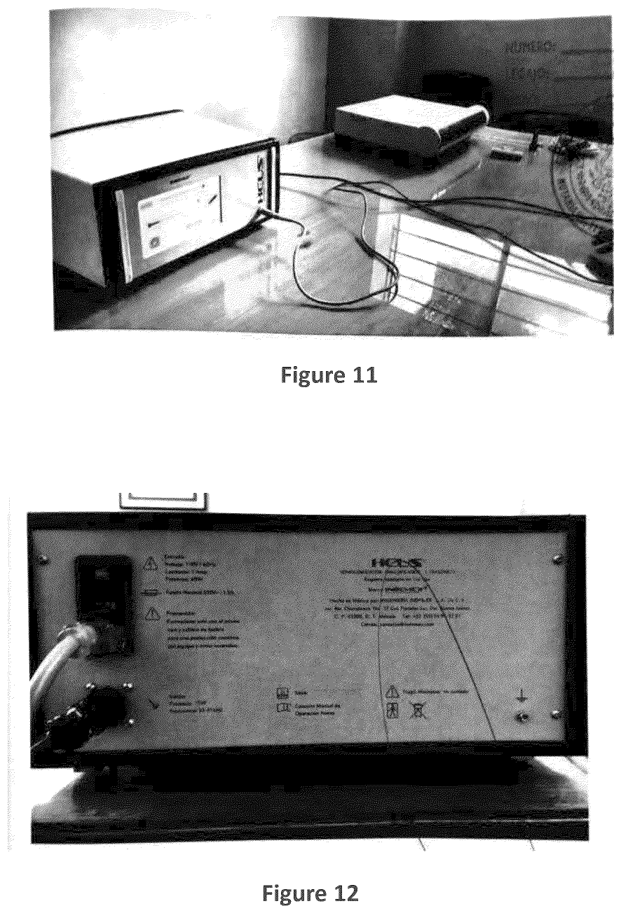

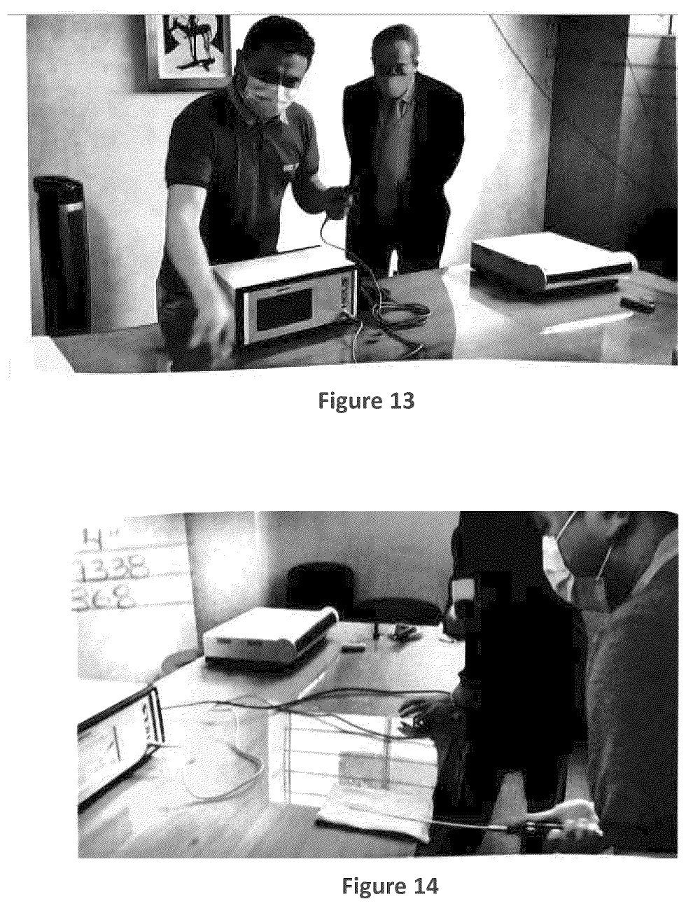

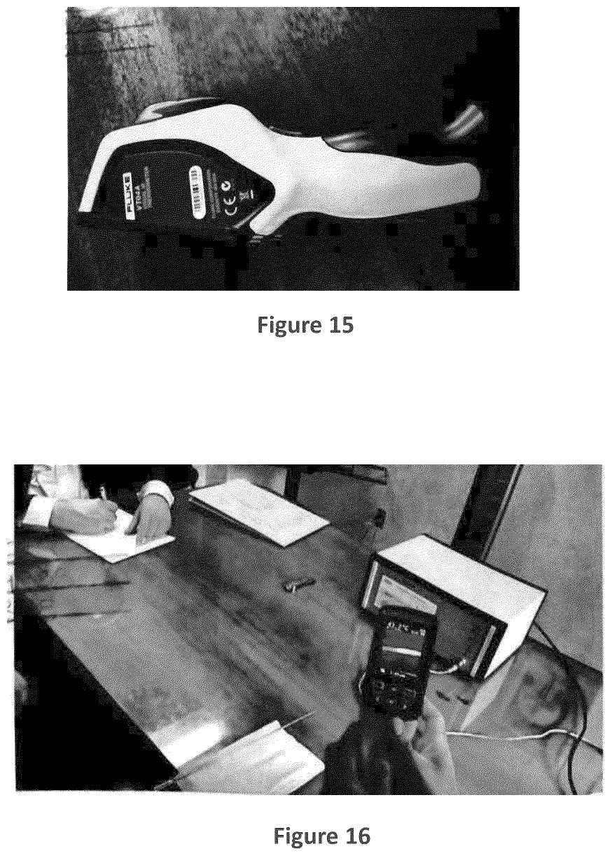

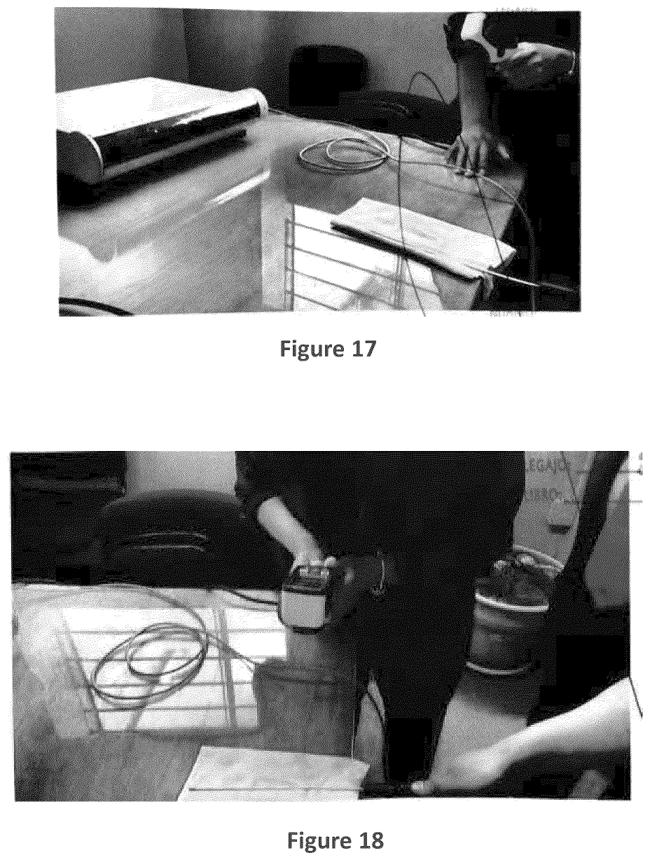

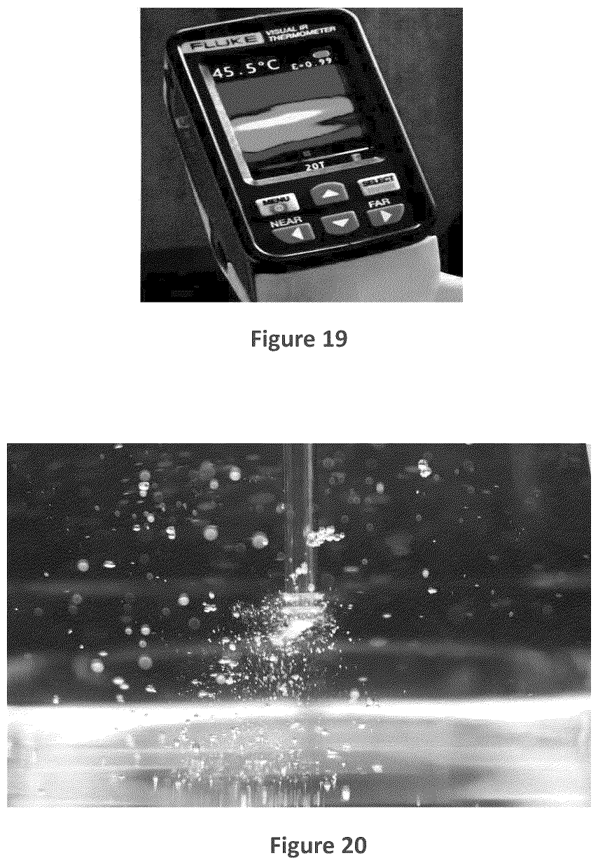

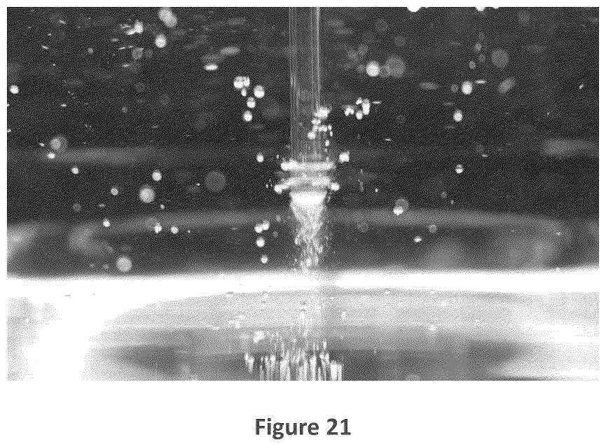

A sonotrode “without rough surface” having a transversal wall in the rings and a longitudinal movement generates active areas, and consequently some active surfaces depending on the number of rings as illustrated in FIG. 9.

ACTIVE AREA=½ SPHERE AREA+S1+S2+S3+S4

A sonotrode “with a rough surface” as it has an area with micro cross-sectional surfaces that, when longitudinally moved, generate a very large active area that optimizes the ultrasound output, as illustrated in FIG. 10.

ACTIVE AREA=Σ of all microsurfaces(Peaks+valleys of the rough area)

In the present invention, where the cannula with a rough tip and constituting a set of additional surfaces has the following advantages:
A greater surface area is offered at different length scales, as shown in FIG. 4.
increases the efficiency of the ultrasound output by means of these scales, as shown in FIG. 4, since the deviations or alterations of the undulation and roughness are found in different orientations, as shown in FIG. 3.
With the optimization of the surface (2) as illustrated in FIG. 3, the tapping zone helps to break the adipocytes, while occupying less energy since the cross-sectional surfaces to the longitudinal movement of the cannula are greater than any smooth surface, it is more stable and uniform. Less power is needed for that output, therefore the sonotrode dissipates less power, that is, it generates less heat.
The thermal impact to the tissue of the human body is reduced, and so, its application reduces the risks of burns and complications in patients undergoing this procedure.
   Comparison of Ultrasonic Emulsifiers with Textured Senotrode and with Smooth Senotrode

### Thermal Impact Reduction

Two devices called ultrasonic emulsifier, one of them HEUSS brand, which according to the inscriptions found on its surface was manufactured in Mexico by “Ingenieria Indemex”, SA de CV, serial number 201907087, and the other device brand “VASER”, which according to the inscriptions found on its surface was manufactured in the United States by “Sound Surgical Technologies LLC” with serial number: VTO4A-20100324, where the rough texture was made. The two teams were connected to the direct current plug located in the same room and immediately afterwards, at fourteen hours and thirty-eight minutes, they connected the Heuss connected to a cable attached to a handpiece or handle, which was attached to a twenty-seven centimeter long titanium cannula, and then the aforementioned device was turned on by operating the power switch located on its rear part, when turning on the device, various images appeared on a screen located on its front part, including a counter or stopwatch at zeros and a power indicator at a level of 100. Then they activated the counter by pressing a pedal or external trigger and immediately began to repeatedly rub the titanium cannula roughly on a previously moistened chamois cloth and placed flat on the same table, this friction action continued until the counter marked one minute, when it was suspended and immediately afterwards they turned on the Fluke model VT04 thermal imaging camera, serial number 20100324, according to the inscriptions attached to its surface, which was placed at a distance of approximately thirty centimeters aligned on the central part of the cannula that had been rubbed and after a few seconds the image of a figure indicating 43.3° C. appeared in the aforementioned chamber and then they proceeded to turn off the device.

Afterwards, a Vaser equipment was attached to a hand piece or handle, which in turn was attached to a twenty-seven centimeter long titanium cannula, and then the aforementioned device was turned on by operating the current switch located on the back, when turning on the device, in some electronic counters that are located on the front, there is a timer at zeros and another counter that indicates the power level at 100. The counter was activated by pressing a pedal or external trigger and immediately afterwards the titanium cannula was rubbed on a previously moistened chamois cloth and placed it flat on the same table, this friction action continued until one minute had elapsed, when it was suspended and turned on the Fluke VT04 thermographic camera again, according to the inscriptions attached to its surface. Which was placed at a distance of approximately 30 centimeters, aligned over the central part of the cannula that had been rubbed, and after a few seconds in the mentioned camera the image of a figure indicating 45.5° C. appeared.

### Operation Methodology

The process for the operation of the smooth surface sonotrode is as follows:
1. Use a 2 liter glass flask.
2. Pour thinner into the flask.
3. Connect the sonotrode to an ultrasonic device at a power of 100.
4. Insert the sonotrode with smooth surface 5. (See FIG. 20).

FIG. 20 shows that when the sonotrode is introduced into the thinner, a dispersed bubbling is formed and there is no concentration of the bubbles, which makes it have a loud sound.

The process for the operation of the rough surface sonotrode is as follows:
1. Use a 2 liter glass flask.
2. Pour Thinner into the flask.
3. Connect the sonotrode to an ultrasonic device at a power of 100.
4. The sonotrode with a rough surface was introduced
5. When introducing the sonotrode to the thinner, a bubble is formed that is located at the tip and goes downwards, which generates a very large active area that optimizes the ultrasound output, which makes it have a low sound as illustrated in the figure twenty-one.

1. An ultrasonic surface optimizer for fragmenting or emulsifying soft tissue in a patient, said optimizer powered by an ultrasonic vibratory energy source, said optimizer comprising:
a longitudinal handle having a proximal end and a distal end;
a longitudinal stem axis joining the proximal end and the distal end;
an axis of the longitudinal stem aligned with the center of the longitudinal stem and passing through the proximal end and the distal end;
a connection at the proximal end of the longitudinal stem for connecting the longitudinal stem to the ultrasonic vibratory energy source;
a point at the distal end of the longitudinal stem; and
a plurality of grooves and rings near the tip, the grooves being generally transverse to the axis, in the axis of the longitudinal shank, and substantially circumscribing the axis of the longitudinal shank, thereby reducing the tissue contact surface area along and around the sides of the tip, providing additional tissue fragmentation surface area of the tip in planes generally perpendicular to the axis; where the grooves and rings of the tip have a rough finish to fragment or emulsify the subcutaneous soft tissues in such a way that a greater surface area is covered in different angular and longitudinal orientations and not only the frontal one.

2. The ultrasonic surface optimizer in accordance with claim 1, characterized in that the rough finish comprises different orientations with a texture in the direction or pattern perpendicular to the direction of the grooves, according to the bisector of the angles formed by the directions of the grooves, in any direction, radial or normal to a radius.

3. The ultrasonic surface optimizer in accordance with claim 1, characterized in that the use of the rough finish due to its nature and its number of micro surfaces at different angles serve to extend the active or output area of the sonotrode, in order to optimize the longitudinal surfaces that are not used in a smooth model or raised only with grooves.

4. The ultrasonic surface optimizer in accordance with claim 1, characterized in that the use of the rough finish is observed by means of a series of peaks and valleys that by their nature show angles along where there is a rough surface, taking all that face of any peak or valley that is arranged transverse to the longitudinal axis of the sonotrode, as an active surface for tapping or ultrasound output.

5. The ultrasonic surface optimizer in accordance with claim 1, characterized in that the use of the rough finish has an area with micro cross-sectional surfaces that generate a very large active area upon longitudinal movement that optimizes the ultrasound output.

6. The ultrasonic surface optimizer in accordance with claim 1, characterized in that the use of the rough finish offers a greater surface area at different length scales.

7. The ultrasonic surface optimizer in accordance with claim 1, characterized in that the rough finish increases the efficiency of the ultrasound output by means of its corresponding scales, since the deviations or alterations of the undulation and roughness are in different orientations.

8. The ultrasonic surface optimizer according to claim 1, characterized in that the use of the rough finish provides optimization of the surface; the tapping zone helps to break down the adipocytes.

9. The ultrasonic surface optimizer in accordance with claim 1, characterized in that the use of the rough finish consumes less energy, because the transverse surfaces to the longitudinal movement of the cannula are greater than any smooth surface, it is more stable and uniform.

10. The ultrasonic surface optimizer in accordance with claim 1, characterized in that the use of the rough finish requires less power for optimal operation, therefore, the sonotrode has less energy dissipation and less temperature radiation.

11. The ultrasonic surface optimizer in accordance with claim 1, characterized in that the use of the rough finish reduces the thermal impact on the tissue of the human body, that is, its application reduces the risks of burns and complications in patients undergoing this procedure.

12. The ultrasonic surface optimizer according to claim 1, characterized in that the plurality of grooves each has a bottom, where each bottom, in a cross section that contains the axis of the longitudinal stem, which is substantially flat and substantially parallel to the longitudinal axis.

13. The ultrasonic surface optimizer according to claim 1, characterized in that each bottom of the plurality of grooves is U-shaped in a cross section that contains the axis of the longitudinal stem.

14. The ultrasonic surface optimizer in accordance with claim 1, characterized in that one or more grooves do not completely circumscribe the axis.

15. The ultrasonic superficial optimizer according to claim 1, characterized in that the longitudinal stem is solid and hollow and has an open lumen along its length, aligned with the axis of the longitudinal stem, and generally located in the center of the longitudinal stem.

16. The ultrasonic surface optimizer according to claim 1, characterized in that the longitudinal stem has a conical section, the conical section is positioned along the axis of the longitudinal stem and between the axis of the longitudinal stem and the distal end of the longitudinal, the conical section is such that the diameter decreases from the longitudinal stem and the distal end of the longitudinal stem.

17. The ultrasonic surface optimizer according to claim 1, characterized in that the tip at the distal end of the longitudinal stem has a blunt or bullet-shaped shape.
